(19) United States
(12) Patent Application Publication
(10) Pub. No.: US 2026/0107961 A1
(43) Pub. Date: Apr. 23, 2026

(54) EDIBLE HYDROGEL MICROCARRIERS COMPRISING CELLS, COMPOSITIONS COMPRISING SAME AND METHODS OF PRODUCING AND USING SAME

(71) Applicant: Sea2Cell Ltd., Kiryat-Shmona (IL)

(72) Inventors: Raziel HAMAMI, Kiryat-Shmona (IL); Orna HAREL, Alonei Abba (IL)

(73) Assignee: Sea2Cell Ltd., Kiryat-Shmona (IL)

(21) Appl. No.: 19/115,277

(22) PCT Filed: May 22, 2024
(86) PCT No.: PCT/IL2024/050502

§ 371 Date: Mar. 26, 2025

Related U.S. Application Data
(60) Provisional application No. 63/468,096, filed on May 22, 2023.
(60) Provisional application No. 63/524,251, filed on Jun. 30, 2023.

(51) Int. Cl. A23L 17/30; A23J 3/06; A23L 29/256; A23L 29/281; A23P 10/30; C12N 5/00; C12N 5/073

(52) U.S. Cl. CPC A23L 17/35; A23J 3/06; A23L 29/256; A23L 29/284; A23P 10/30; C12N 5/0012; C12N 5/0603; C12N 2513/00; C12N 2531/00

(57) ABSTRACT

A composition comprising edible hydrogel microcarriers-comprising cells, wherein said cells are distributed throughout said hydrogel microcarriers such that said hydrogel microcarriers are devoid of a cell-free core. Also provided are methods or producing and using same.

### RELATED APPLICATIONS

This application claims the benefit of priority from U.S. Provisional Patent Application Nos. 63/468,096, filed on May 22, 2023 and 63/524,251 filed Jun. 30, 2023 each of which is incorporated herewith in its entirety.

### FIELD AND BACKGROUND OF THE INVENTION

The present invention, in some embodiments thereof, relates to edible hydrogel microcarriers comprising cells, compositions comprising same and methods of producing and using same.

Tissue engineering is an emerging field that aims at creating complex tissues from cells. Fish tissue engineering is a rapidly growing field that has the potential to revolutionize aquaculture and fisheries management. Fish are an important source of protein for human consumption and play a key role in the ecosystem. However, the demand for animal protein is expected to increase significantly in the coming years due to population growth and changes in dietary preferences. Yet, the current methods of animal protein production, such as intensive farming and fishing, have a significant environmental impact and raise ethical concerns.

Cultivated meat, also known as cell-based meat, offers a promising alternative as it has the potential to produce animal protein with fewer environmental impacts and without the need for animal slaughter. The development of cultivated fish meat could have significant benefits for both the environment and human health. However, its development and commercialization require addressing several technical challenges, including the ability to scale up cell production and create tissue-like structures with desirable organoleptic properties. One of the key approaches to tissue engineering is the use of hydrogel matrices, which provide a supportive 3D environment for cell proliferation and differentiation [Levenberg et al. (2003). Proceedings of the National Academy of Sciences, 100 (22), 12741-12746].

Hydrogels are a class of polymeric materials that possess a high-water content and a three-dimensional (3D) network structure. They are composed of hydrophilic polymers crosslinked to form a gel-like structure that swells but does not dissolve in water. Hydrogels have gained significant attention in various fields, including tissue engineering, due to their unique properties and potential applications. They also play a crucial role in cell encapsulation, particularly in the context of creating 3D cell-seeded microcarriers. Cell-seeded microcarriers are small particles or beads designed to support cell growth and organization. They serve as carriers for cells, providing a substrate for cell attachment, proliferation, and differentiation in a 3D hydrogel environment.

Compared to traditional scaffold-based approaches, microcarriers offer several distinct advantages. Firstly, microcarriers have a larger surface area-to-volume ratio, allowing for increased cell attachment and growth. This property is particularly beneficial when working with limited cell numbers or when aiming to scale up cell production. Secondly, microcarriers facilitate improved nutrient and oxygen penetration throughout the cell population due to their smaller size and increased surface area. This enhanced mass transfer promotes cell viability and function within the construct. Additionally, microcarriers offer enhanced scalability and ease of handling, allowing for efficient and controlled cell expansion in bioreactor systems. These features make microcarriers a valuable tool in tissue engineering, enabling the generation of functional tissue constructs with improved cell growth and organization.

Among the different types of hydrogels, polysaccharide-based hydrogels have emerged as a promising option. Polysaccharides are natural carbohydrates composed of repeating sugar units. They offer several advantages for hydrogel formation and cell encapsulation. Two commonly used polysaccharides in 3D cell culturing are chitosan and alginate [Suh et al. 2000 Biomaterials, 21 (24), 2589-2598]. Chitosan, derived from chitin found in the exoskeleton of crustaceans, is a biocompatible and biodegradable polysaccharide. Alginate, derived from brown seaweed, stands out as one of the most favored materials for producing cell-seeded microcarriers due to its exceptional properties. As an extensively used polysaccharide hydrogel, alginate offers a range of advantages that make it ideal for cell encapsulation.

Unlike mammalian cells, fish cells thrive in cooler environments, necessitating a specialized approach for creating fish cell-seeded microcarriers.

Additional background art includes:
Rubio et al. Front. Sustain. Food Syst., 11 Jun. 2019 Sec. Nutrition and Sustainable Diets; www (dot) frontiersin (dot) org/articles/10 (dot) 3389/fsufs (dot) 2019 (dot) 00043/full
U.S. Pat. No. 9,752,122
U.S. Pat. No. 10,208,281
Cohen et al. Biomaterials 295 (2023) 122033
Baruch and Machluf Biopolymers, Vol. 82, 570-579 (2006)
West et al. Biomaterials 28 (2007) 4439-4448
Norris et al. Biomaterials 287 (2022) 121669
Saltz et al. Biomed Mater Res Part A 2016: 104A: 1276-1284

### SUMMARY OF THE INVENTION

According to an aspect of some embodiments of the present invention there is provided a composition comprising edible hydrogel microcarriers-comprising cells, wherein the cells are distributed throughout the hydrogel microcarriers such that the hydrogel microcarriers are devoid of a cell-free core.

According to an aspect of some embodiments of the present invention there is provided a method of producing edible hydrogel microcarriers-comprising cells, the method comprising:
(a) contacting a first solution comprising a cross-linker and a second solution comprising a polysaccharide, wherein at least one of the first solution and the second solution comprises cells so as to obtain a cross linked hydrogel comprising the cells;
(b) forming the hydrogel microcarriers-comprising cells from the cross linked hydrogel, such that the microcarriers are devoid of a cell-free core.

According to an aspect of some embodiments of the present invention there is provided a composition comprising edible hydrogel microcarriers-comprising cells obtainable according to the method as described herein.

According to an aspect of some embodiments of the present invention there is provided a method of producing a cell line, the method comprising passaging in a culture medium the composition as described herein, which comprises heterogeneous cells to obtain a cell line.

According to an aspect of some embodiments of the present invention there is provided a method of culturing cells, the method comprising incubating the composition as described herein in culture under conditions which allow proliferation and/or differentiation of the cells, thereby culturing the cells.

According to some embodiments of the invention, the hydrogel microcarriers are composed of a cross-linked polysaccharide.

According to some embodiments of the invention, the polysaccharide is natural.

According to some embodiments of the invention, the polysaccharide is synthetic.

According to some embodiments of the invention, the polysaccharide is biodegradable.

According to some embodiments of the invention, the polysaccharide is biocompatible.

According to some embodiments of the invention, the polysaccharide is selected from the group of saccharides listed in Table 1.

According to some embodiments of the invention, comprising a protein or peptide which serves as a structural protein composing the microcarrier.

According to some embodiments of the invention, the composition is edible.

According to some embodiments of the invention, the protein is gelatin.

According to some embodiments of the invention, the protein or peptide is RGD-containing.

According to some embodiments of the invention, the polysaccharide is alginate.

According to some embodiments of the invention, the polysaccharide is alginate and the protein is gelatin.

According to some embodiments of the invention, the method further comprises removing the polysaccharide following the forming the edible hydrogel microcarriers-comprising cells.

According to some embodiments of the invention, the removing is by using a chelator, e.g., EDTA or acid, e.g., citric acid.

According to some embodiments of the invention, the microcarriers are of an average size range of 200-1500 μm or 500-1500 μm.

According to some embodiments of the invention, a diameter of the cell-free core does not exceed 1500 μm.

According to some embodiments of the invention, the cells comprise 1 or 2 cell types.

According to some embodiments of the invention, the cells are of an aquatic species.

According to some embodiments of the invention, the cells are fish cells.

According to some embodiments of the invention, the fish cells are of a fish species selected from a group comprising of salmon, grouper, tuna, snapper, mackerel, cod, trout, carp, catfish, shark and sardine.

According to some embodiments of the invention, the cells are of an avian or a mammalian species.

According to some embodiments of the invention, the cells are stem cells.

According to some embodiments of the invention, the cells are myogenic cells.

According to some embodiments of the invention, the cells are adipocytes, muscle cells, blood cells, cartilage cells, bone cells, connective tissue cells, fibroblasts, cardiomyocyte, hepatocytes, cardiomyocyte, epithelial cell and/or gill cells

According to some embodiments of the invention, the forming is by microfluidics.

According to some embodiments of the invention, the forming is by 3D printing.

According to some embodiments of the invention, the contacting is concomitantly with the forming.

According to some embodiments of the invention, the forming is by microfluidics.

According to some embodiments of the invention, the contacting is prior to the forming.

According to some embodiments of the invention, the edible composition comprises an additive selected from the group consisting of one or more of: are color enhancers, taste enhancers, nutritional additives, fiber, carbohydrate, meal, preservatives, fats and/or oils.

According to some embodiments of the invention, the additive is selected from the group consisting of an Omega 3, Omega 6, Fatty acid, Vitamin D, Vitamin A, Taurine, amino acid, dietary fiber and an antioxidant.

According to some embodiments of the invention, the culturing is in a bioreactor.

According to some embodiments of the invention, the culturing is in the presence of an exogenously added growth factor.

According to some embodiments of the invention, the culturing is in a suspension culture.

According to some embodiments of the invention, the cells form a microsphere in the microcarrier.

According to some embodiments of the invention, the cells form filopodia.

According to some embodiments of the invention, the composition or the hydrogel microcarriers are animal free.

According to an aspect of some embodiments of the present invention there is provided a cultured meat comprising the composition as described herein.

According to an aspect of some embodiments of the present invention there is provided a comestible comprising the cultured meat composition as described herein.

According to some embodiments of the invention, the meat composition is selected from a group comprising a cake, a ball, a burger, a canned composition, an imitation of a steak, a stick, a ground meat, a nugget and a sausage.

According to some embodiments of the invention, the comestible is processed to impart an organoleptic sensation and texture of meat.

According to some embodiments of the invention, the comestible further comprises other comestible additives.

According to some embodiments of the invention, the comestible further comprises a plant and/or animal-originated food stuffs and/or plant and/or animal-based proteins.

According to some embodiments of the invention, the comestible further comprises a plant-based protein.

According to an aspect of some embodiments of the present invention there is provided a method of producing food, the method comprising combining the cultured meat as described herein or the comestible as described herein with an edible composition for human or animal consumption.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWING(S)

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is an image of AGG cell-seeded microcarriers with 1×10⁶ STE-137 cells/ml on day 1. Scale bar: 100 μm.

FIGS. 2A-B show cell branching and filopodia formation on day 7 (A) and 14 (B). Scale bar: 100 μm.

FIG. 3 shows STE-137 cells migration to the petri dish surface on day 3 (black arrow). Scale bar: 100 μm.

FIG. 4 shows actin filaments visualized in encapsulated STE-137 cells on day 7. Fluorescent staining was performed with Phalloidin (red) for F-actin and DAPI (blue) for nuclei. Images were captured using a fluorescence microscope. Scale bar: 100 μm.

FIG. 5A shows microscopy images of STE-137 spheroids formation in AGG microcarriers on day 7. Panel A—Brightfield images of STE-137 spheroids in AGG microcarriers. Panel B—Fluorescence images of STE-137 spheroids stained with phalloidin (red) and DAPI (blue). Scale bar: 100 μm.

FIG. 5B shows microscopy images of STE-137 spheroids formation in AGG microcarriers on day 14. Panel A—Brightfield images of STE-137 spheroids in AGG microcarriers. Panel B—Fluorescence images of STE-137 spheroids stained with phalloidin (red) and DAPI (blue). Scale bar: 100 μm.

FIG. 6 shows microscopy images of STE-137 cells migration from the microcapsule to the petri dish on day 25. Scale bar: 100 μm.

FIG. 7 shows STE-137 spheroids on day 40. Scale bar: 100 μm.

FIGS. 8A-B show microscopy images of Gilthead Bream (GHB) cells (FIG. 8A) 1 day (left panel) or 5 days following encapsulation in alginate-gelatin capsules (AGG); and similarly, encapsulated Rainbow Trout (RT) ovary cells (FIG. 8B). Scale bars indicate 0.5 millimeters.

### DESCRIPTION OF SPECIFIC EMBODIMENTS OF THE INVENTION

The present invention, in some embodiments thereof, relates to edible hydrogel microcarriers comprising cells, compositions comprising same and methods of producing and using same.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Whilst conceiving and reducing to practice embodiments of the invention, the present inventors have devised a novel microcarrier configuration in which the cells are spread throughout the microcarrier without leaving a cell-free core. This configuration is expected to be tolerant to hypoxia, have high buffering capacity and withstand low-temperature growth conditions which is typically required for marine-based cells, and protect cells from shear forces when grown in a bioreactor. Taken together this configuration makes marine cell culture an attractive tool for scaled production of cell-based seafood perhaps, even more than mammalian and avian cells.

Embodiments of the invention make use of a polysaccharide matrix, in particularl alginate, to support the growth and adaptation of fish cell cultures and cell lines to grow in suspension systems and promote cultured meat applications. In certain embodiments, the present inventors used alginate alone or a mixture of alginate and gelatin to create a hydrogel matrix, which was subsequently used to encapsulate STE-137 cells, RT ovary cells (both being from Rainbow Trout) or Gilthead Seabream (GHB). The cells were cultivated for more than 30 days (even 50 days), to form 3D-spheroids inside the capsules. The cell-seeded microcarriers were transferred every 3 days to a new cell culture dish to facilitate cell migration and the formation of new adapted cell lines. Specifically, the cells exhibited migration from the particles to the dish and adherence to the dish. Such cells were later removed from the dish and introduced back to the microcarriers, where they were better adapted to 3D growth in the hydrogel. Actin formation in the cells was evaluated using phalloidin fluorescence staining. Migration of cells and formation of filopodia were observed in these settings. Filopodia are essential structures that cells use to explore and interact with their surrounding environment. Filopodia extend from the leading edge of cells and make contact with other cells, the extracellular matrix, or other surrounding structures, such as natural or synthetic matrices, e.g., scaffolds. This contact initiates a cascade of intracellular signaling events that ultimately determine the fate and behavior of the cell. In a 3D setting, filopodia play a critical role in the formation of functional tissue structures [Jacquemet et al. (2015) Current opinion in cell biology, 36, 23-31]. They facilitate the communication between cells and enable the exchange of signaling molecules, nutrients, and waste products [Abounit, & Zurzolo (2012). Journal of cell science, 125 (5), 1089-1098]. The formation of filopodia in the present experiment suggests that the encapsulated fish cells are actively exploring their surrounding environment and interact with the polysaccharide matrix, which could lead to the formation of functional tissue structures.

These results demonstrate the potential of the polysaccharide matrix of some embodiments of the invention, for tissue engineering applications, including the development of cultured meat and in particular fish meat.

Hence, fish cell encapsulation and the utilization of fish cell-seeded microcarriers in bioreactors and suspension cultures offer promising prospects for the advancement of cultivated seafood production. The use of hydrogel matrices provides a conducive 3D environment for fish cell growth and differentiation, facilitating the formation of spheroids. The utilization of microcarriers in biorcactors, along with the incorporation of controlled extracellular matrix (ECM), enables scalable cell production and provides precise regulation of nutrient supply and waste removal. Cell-seeded microcarriers, with their larger surface area for cell attachment and growth, serve as a platform for cells to interact with a tailored ECM environment. By integrating the ECM components into the microcarrier structure, such as incorporating specific proteins or signaling molecules, the culture conditions can be fine-tuned to mimic the natural cellular microenvironment. This controlled ECM environment enhances cell adhesion, proliferation, and differentiation, leading to improved cell yield and functionality. The combination of microcarriers and controlled ECM within bioreactors offers a powerful approach for achieving efficient and high-quality cell production for various tissue engineering and regenerative medicine applications. As mentioned, cells that survived the microcarriers culturing conditions and maintained proliferation capacity in 2D (e.g., on a petri dish), are more prone to adapt to 3D growth conditions. This technology has the potential to revolutionize the sustainable production of seafood on a large scale, alleviating the pressure on natural fish populations and marine ecosystems.

Thus, according to an aspect of the invention there is provided a composition comprising edible hydrogel microcarriers-comprising cells, wherein said cells are distributed throughout said hydrogel microcarriers such that said hydrogel microcarriers are devoid of a cell-free core.

As used herein “microcarriers” refer to fine particles which are slightly heavier than water which allow culturing the cells in a suspended state.

As opposed to standard microcarrier cultures in which the cultivation of cells in a suspended state occurs by proliferating cells as a layer on the surface of the microcarrier (leaving a cell-free core), in the present case, the cells are distributed throughout the carrier and not on the surface.

According to some embodiments of the invention, the microcarrier is a spherical particle having a diameter of 500-1500 μm or 500-1500 μm, e.g., 500-1000 μm, 1000-1500 μm.

According to some embodiments, the diameter is between 0.02-3 mm, 0.02-2 mm, 0.02-1 mm, 0.05-3, 0.05-2 mm, 0.05-1 mm. 0.1-3 mm, 0.1-2 mm. 0.1-1 mm, 0.5-1 mm. 0.5-2 mm, 0.5-3 mm.

When relating to a specific measure, it means an average measure in a given preparation, for example the average particle diameter.

According to some embodiments of the invention, the microcarrier has a surface area of 100-1000 cm²/g.

According to some embodiment, the microcarrier has a specific gravity 1.01-1.2 g/cm³.

According to a specific embodiment, essentially all the microcarriers in the culture/preparation comprise cells (since they are included in the fabrication process). In stark contrast, when cell seeding is involved (prior art microcarriers) not all the microparticles will comprise cells either because of no cell seeding or because the cells have detached from the microcarriers.

As used herein “distributed throughout” means that the concentration of the cells is about the same at every point in the microcarrier. In other words, there are no areas within the volume where the concentration of the cells is significantly higher or lower compared to other areas, especially not in the center of the microcarrier. The center of the carrier is referred to as “a core” and according to some embodiments of the invention it comprises viable cells. This can be assayed using brightfield microscopy as shown in the Examples section which follows. A uniform appearance means that the cells are evenly dispersed or deposited through the entire volume. It will be appreciated that the cells may form clusters or spheres within the sphere, In such a case there is still a uniform distribution throughout the matrix, which is not essentially restricted to the surface as in coating methods.

Hence, distributed throughout may also mean that the cells are not essentially restricted to the surface or subsurface of the particle (as is the case in coating methods).

As used herein the term “hydrogel” describes a three-dimensional fibrous network containing at least 20%, typically at least 50%, or at least 80%, and up to about 99.99% (by mass) water. A hydrogel can be regarded as a material which is mostly water, yet behaves like a solid or semi-solid due to a three-dimensional crosslinked solid-like network, made of natural and/or synthetic polymeric chains, within the liquid dispersing medium.

The phrases “aqueous solution” and “aqueous medium” are used interchangeably.

As used herein, the phrase “fibrous network” refers to a set of connections formed between the polymers in the preparation from which the particle is composed e.g., polysaccharides and optionally protein. The components of the hydrogel particles are further described hereinbelow in the context of the method but these apply to the composition per se too.

According to a specific embodiment, the particles are subjected to lyophilization.

Accordingly, there is provided a preparation comprising lyophilized particles. The term “lyophilized” or “freeze-dried” includes a state of a substance that has been subjected to a drying procedure such as lyophilization, where at least 50% of moisture has been removed.

According mentioned, the composition is composed of an edible hydrogel.

As used herein “edible” refers to a composition which is safe for human or animal eating. For example, this includes, but is not limited to a food product that is generally recognized as safe per a government or regulatory body (such as the United States Food and Drug Administration). In certain embodiments, the food product is considered safe to consume by a person of skill. Any edible food product suitable for a human consumption should also be suitable for consumption by another animal and such an embodiment is intended to be within the scope herein.

According to a specific embodiment, the composition is animal-free, i.e., does not comprise any substance which is isolated from an animal body (excluding the cells).

As used herein “food grade” refers to a substance which is either safe for human consumption or confirmed to come into direct contact with food products.

According to some embodiments of the invention, the materials used in the context of the invention are of “food grade” classification.

Such substances are also referred to herein and in the art as “food contact substances” or “food contact materials”.

The phrase “food contact substance” or FCS, is used herein to describe substances that are generally safe for human consumption by virtue of being generally recognized as safe (GRAS) or by passing standard safety tests, and thus qualify for use as a component of materials used in manufacturing, packing, packaging, transporting, or holding food, in the same manner it is meant in the guideline and regulation of worldwide food administration authorities, such as, for example, the U.S. Food and Drug Administration (FDA), Center for Food Safety and Applied Nutrition (CFSAN), the Office of food Additive Safety.

The phrase “generally recognized as safe” or GRAS, as used herein, is meant in the same manner which is defined, for example, under sections 201 (s) and 409 of the U.S. FD&C Act. The U.S. law states that any substance that intentionally contacts food or added to food is a food additive, that is subject to premarket review and approval by FDA, unless the substance is generally recognized, among qualified experts, as having been adequately shown to be safe under the conditions of its intended use, or unless the use of the substance is otherwise excluded from the definition of a food additive. GRAS substances are distinguished from food additives by the type of information that supports the GRAS determination, that it is publicly available and generally accepted by the scientific community, but should be the same quantity and quality of information that would support the safety of a food additive. Since the qualification to an FCS or GRAS category can be obtained through a process of applying, testing and qualifying to the requirements of the various official food and drug authorities, the present embodiments are meant to encompass all relevant substances and their derivatives which are to become FCSs and GRAS in the future, as well as those which already qualify as FCSs and GRAS.

According to a specific embodiment, the hydrogel microcarriers comprise a polysaccharide (e.g., at least one type) and optionally a protein (e.g., at least one type).

According to a specific embodiment, the hydrogel microcarriers are composed of a polysaccharide, e.g., cross-linked polysaccharide.

According to a specific embodiment, the polysaccharide is edible.

Polysaccharides are constructed from monomeric sugars that are linked together by O-glycosidic linkages. According to some embodiments, the polysaccharide is selected biocompatible and can be purified from nature or synthetically synthesized.

Thus, according to some embodiments, the polysaccharide is naturally occurring.

According to alternative or additional specific embodiments, the polysaccharide is synthetic.

The latter option allows including some modifications which will increase its bioavailability, safety, improved mechanical properties or co-formulation properties with the protein.

Non-limiting examples are provided infra.

Cellulose-Cellulose is a type of polysaccharide found abundantly in nature and is easily chemically modified, which provides many advantages. Cellulose forms the structural basis in plants, which makes it the most abundant renewable resource on the planet. As a biomaterial, cellulose has served as wound dressings and in the form of hydrogels for orthopedic applications. Favorable properties include high tensile strength and biocompatibility. Different means of enhancing its properties has been explored, such as phosphorylation or bacterial synthetization, which increase its bioactivity.

Chitin and Chitosan-Chitin serves as a major structural component of invertebrates, insects, and fungi. It is an extremely abundant biopolymer, right after cellulose. In its purest form, it is insoluble in water. Its structure is a highly linear and it is a highly crystalline polymer. The material chitosan can be found in a few fungi species, and is mainly produced through chitin deacetylation. Due to its high degree of crystallinity, the materials are extremely stable through hydrogen bonding. These materials contain no antigenic properties, which makes them biocompatible as well as eco-friendly.

Starch—Starch is an abundant polysaccharide that is found in the roots, seeds, and stems of various plants and crops. Starch is constructed of two different polymers: Amylose and amylopectin. While starch presents a few disadvantages, such as low mechanical strength and high hydrophilicity, it has demonstrated good biodegradability and cell seeding capabilities. Therefore, starch has excellent structural capabilities for biodegradability and biocompatibility. Starch is relatively easy to modify, which makes it suitable for chemical enhancements to improve upon its weaker qualities.

Pectin—Pectin is a carbohydrate material derived from plant walls, mainly as a citrus byproduct. Pectin has excellent gelation properties. It is also hydrophilic in nature with many functional capabilities. It can be divided into three main regions: Smooth, hairy, and branched. The gelling property as well as solubility is dependent upon the esterification of galacturonic acid residues. Because of its gel-forming abilities, it has been recommended for the use of delivery bioactive agents. Pectin is non-toxic, and high in fiber content, which has made it successful in the food industry. Alginates-Alginates are an important polysaccharide and can be found in algae species and soil bacteria. Being one of the most biosynthesized materials, alginates are naturally hydrophilic and anionic. Alginates have an excellent ability to store and retain water, as well as stabilizing and gelation properties. Chelation properties also make alginates favorable in drug delivery systems or tissue regeneration.

Hyaluronic Acid-Hyaluronic acid is a natural linear polysaccharide found in the extracellular matrix of animals. This material is naturally biocompatible, biodegradable, and lacks immunogenicity. Its structural properties give it the ability to mediate cell signaling, provide wound repair, and declare matrix organization.

Also contemplated herein are derivatives of these polysaccharides such as in the case of cellulose, e.g., carboxymetyl cellulose, methyl cellulose or hydroxypropyl cellulose.

Other polysaccharides include but are not limited to dextrin, carrageenan, gum Arabic, and xanthan gum.

According to a specific embodiment the polysaccharide is alginate.

As mentioned, the polysaccharides and/or protein can be naturally occurring. Thus, in some embodiments, it may be extracted from a natural source such as a plant, an animal, a sponge or a coral.

Soft corals-based gels are taught in Hassan et al. Pharmaceuticals 2023, 16 (7), 957, discussing specifically, A Nephthea sp. methanol-methylene chloride extract loaded with pectin nanoparticles (LPNs) to which cells can be added such as by using ion-gelation techniques.

Sponges based gels are taught in Wang et al. Chemical Eng. J. Volume 427, 1 Jan. 2022, 130905.

Plant-based hydrogels such as from succulents and cacti are taught in Kamal et al. Gels 2022, 8(2), 88 (a composite of bacterial cellulose and a hydrogel from cactus); hydrogel from Aloe Vera is taught in Jales et al. J. Compos. Sci. 2022, 6 (8), 231; and in Drabczyk et al. Materials 2020, 13(14), 3073 (a composite of Avera and Chitosan);

Seaweed as a source for cellulose is described in Bar-Shai et al. Scientific Reports volume 11, Article number: 11843 (2021); and in Lee et al. Food Hydrocolloids Volume 152, July 2024, 109944; Barre et al. teach lectins for hydrogel production see Mar. Drugs 2019, 17(8), 440, each of which is incorporated herein in its entirety.

TABLE 1
This table provides an overview of some commonly used polysaccharide
hydrogels, including their sources and properties. Each polysaccharide
offers unique characteristics that make them suitable for specific
applications, highlighting the versatility and potential of polysaccharide
hydrogels in the realm of biomaterials.

| Polysaccharide | Source | Properties |
|---|---|---|
| Chitosan | Derived from chitin | Biocompatible, |
| | | biodegradable, |
| | | antimicrobial |
| Alginate | Derived from brown seaweed | Low toxicity, |
| | | gelation with |
| | | divalent cations |
| Agarose | Derived from seaweed | Temperature-induced |
| | | gelation, porous |
| | | structure |
| Hyaluronic | Naturally occurring | Biocompatible, |
| Acid (HA) | | water retention |
| Cellulose | Found in plant cell walls | Tunable properties, |
| | | mechanical strength |
| Dextran | Produced by bacteria | Controlled porosity, |
| | | swelling properties |
| Xanthan Gum | Produced by bacteria | Shear-thinning behavior |
| Pectin | See U.S. Pat. No. 9,752,122 | |

The concentration range of the polysaccharide e.g., alginate (and gelatin when added) in the composition can vary depending on the specific requirements of the (e.g., fish) cell-seeded microcarriers. The optimal composition will depend on factors such as the desired physical properties of the hydrogel, cell type, and culture conditions.

According to a specific embodiment, for alginate, the concentration typically ranges from 0.5% to 4% (w/v) in the hydrogel formulation, e.g., 1-2%, 0.5-3%, 0.5-2%, 0.5-1%, 0.5-2.5%, 0.8-2%, 0.8-1%, 0.8-1.5%, 0.8-2.5%, 0.8-3%, 0.8-3.5%, 1-4%, 1-3%.

For example, for alginate the concentration can be 0.5%-4% (w/v) in the hydrogel.

According to an alternative or an additional embodiment, the concentration of gelatin in the hydrogel is 0.5-5%.

According to a specific embodiment, the polysaccharide is biodegradable.

According to a specific embodiment, the polysaccharide is biocompatible.

As used herein, the term “biocompatible” refers to the ability of a material to be compatible with living tissues or biological systems without causing harm or adverse reactions. A biocompatible material is designed to interact with biological systems in a way that does not elicit a toxic, immunological, or inflammatory response.

As used herein, the term “biodegradable” refers to a substance that can be broken down or decomposed into simpler compounds by the action of living organisms, such as bacteria, fungi, or enzymes. These materials undergo degradation over time, eventually converting into smaller molecules that can be absorbed or assimilated by the environment without causing harm.

According to some embodiments, the composition comprises a protein (e.g., gelatin) or peptide which serves as a structural protein composing said microcarrier.

The term “peptide” as used herein encompasses native peptides (either degradation products, synthetically synthesized peptides or recombinant peptides) and peptidomimetics (typically, synthetically synthesized peptides), as well as peptoids and semipeptoids which are peptide analogs, which may have, for example, modifications rendering the peptides more stable while in a body or more capable of penetrating into cells. Such modifications include, but are not limited to N terminus modification, C terminus modification, peptide bond modification, backbone modifications, and residue modification. Methods for preparing peptidomimetic compounds are well known in the art and are specified, for example, in Quantitative Drug Design, C.A. Ramsden Gd., Chapter 17.2, F. Choplin Pergamon Press (1992), which is incorporated by reference as if fully set forth herein. Further details in this respect are provided hereinunder.

Peptide bonds (—CO—NH—) within the peptide may be substituted, for example, by N-methylated amide bonds (—N(CH3)-CO—), ester bonds (—C(═O)—O—), ketomethylene bonds (—CO—CH2-), sulfinylmethylene bonds (—S(═O)—CH2-), α-aza bonds (—NH—N(R)—CO—), wherein R is any alkyl (e.g., methyl), amine bonds (—CH2-NH—), sulfide bonds (—CH2-S—), ethylene bonds (—CH2-CH2-), hydroxyethylene bonds (—CH(OH)—CH2-), thioamide bonds (—CS—NH—), olefinic double bonds (—CH═CH—), fluorinated olefinic double bonds (—CF═CH—), retro amide bonds (—NH—CO—), peptide derivatives (—N(R)—CH2-CO—), wherein R is the “normal” side chain, naturally present on the carbon atom.

These modifications can occur at any of the bonds along the peptide chain and even at several (2-3) bonds at the same time.

For example, natural aromatic amino acids, Trp, Tyr and Phe, may be substituted by non-natural aromatic amino acids such as 1,2,3,4-tetrahydroisoquinoline-3-carboxylic acid (Tic), naphthylalanine, ring-methylated derivatives of Phe, halogenated derivatives of Phe or O-methyl-Tyr.

The peptides of some embodiments of the invention may also include one or more modified amino acids or one or more non-amino acid monomers (e.g. fatty acids, complex carbohydrates etc).

The term “amino acid” or “amino acids” is understood to include the 20 naturally occurring amino acids; those amino acids often modified post-translationally in vivo, including, for example, hydroxyproline, phosphoserine and phosphothreonine; and other unusual amino acids including, but not limited to, 2-aminoadipic acid, hydroxylysine, isodesmosine, nor-valine, nor-leucine and ornithine. Furthermore, the term “amino acid” includes both D- and L-amino acids.

The peptides of some embodiments of the invention may be synthesized by any techniques that are known to those skilled in the art of peptide synthesis. For solid phase peptide synthesis, a summary of the many techniques may be found in J. M. Stewart and J. D. Young, Solid Phase Peptide Synthesis, W. H. Freeman Co. (San Francisco), 1963 and J. Meinhofer, Hormonal Proteins and Peptides, vol. 2, p. 46, Academic Press (New York), 1973. For classical solution synthesis see G. Schroder and K. Lupke, The Peptides, vol. 1, Academic Press (New York), 1965. Recombinant techniques are also contemplated where possible.

As mentioned the protein component is preferably edible. Examples of such proteins include, but are not limited to collagen, gelatin, fibronectin, pea protein, soy protein, whey protein and casein. As mentioned, also contemplated herein are fragments and modifications thereof.

According to a specific embodiment, the protein is gelatin. Such a configuration is described in the Examples section which follows.

According to a specific embodiment, the protein or peptide is RGD-containing.

The RGD sequence consists of three amino acids: arginine (R), glycine (G), and aspartic acid (D). This sequence is a common motif found in various proteins and peptides, particularly in extracellular matrix proteins like fibronectin, vitronectin, laminin, and collagen. RGD is recognized by integrin receptors, specifically integrin αvβ3, which are present on the surface of many cell types, including endothelial cells and smooth muscle cells. When cells encounter the RGD sequence, it can trigger signaling pathways that influence cell behavior, such as adhesion, migration, proliferation, and differentiation facilitating in the culturing of the cells on the microcarriers.

Examples of RGD peptides comprise, GRGDSP, CRGDS, KLTGRGDS, GRGDSPC, RGD-SP. Other examples are known in the art. The peptides can be longer, such as naturally occurring or in some other embodiments shorter than the naturally occurring protein or peptide which comprise same.

For example, one class of RGD containing protein that may be used includes the cardosins. Cardosins are aspartic proteinases that may be extracted from Cynara cardunculus L., and that contain cell binding RGD motifs that promote cell attachment. For example, cardosins may be derivatized through their cysteines to introduce new thiol groups. Cardosins are already used by the food industry, specifically in cheese-making, (see, e.g., www(dot)culturecheesemag(dot)com/ask_the_monger/vegetarian_cheese).

According to a specific embodiment, the polysaccharide is alginate and the protein is gelatin. Such an embodiment is described in the examples section which follows and is referred to as AGG.

According to a specific embodiment, the protein to polysaccharide molar ratio is between 1:1 and 1:5, e.g., 1:1-1:3, 1:1-1:2 (e.g., gelatin to alginate that can be used as a reference for other combinations).

The microcarriers may also comprise a cross linker (e.g., edible) which serves in the hydrogel production process, as will be described in details below.

Other than that the composition may comprise components which are compatible with cell culturing and even promote it. These include, growth factors, vitamins, antibiotics, differentiation factors and the like. See e.g., Mishra et al., 2018 Hydrogels: Recent Advances Chapter 6 Preparation, Properties and Application of Hydrogels: A Review.

The composition comprising the microcarriers may include any additive (such as mentioned below and/or in the following list; Thus, for example, a flavoring, a flavor enhancer, a colorant, a color enhancer, and/or a nutritional enhancer. Any known flavoring/flavorant, or combination of these, may be used, including meat flavors: such as pork (e.g., 2-pyridine methanethiol), chicken, beef, veal, turkey, lamb, etc.: fat and oil flavors (e.g., flavors corresponding one or more of: fried fat, lard, tallow, chicken fat, bacon fat, turkey fat, pork fat, beef fat, sesame oil, olive oil, etc.); dairy flavors (cheese, cream, milk, sour cream, etc.) and the like. See, e.g., US-2014-0205729, herein incorporated by reference in its entirety.

Stiffness and porosity features may be controlled by the selection of the polymers and cross-linkers, thereby governing cell behavior and functionality.

It will be appreciated that the selected components and the cross-linker(s) and methods of producing the microcarriers will affect the ability of the cells to withstand certain growth conditions such as low temperatures e.g., 18-30° C., 18-25° C., 18-22° C. 18-20° C. or below 20° C., which is of significance for marine species.

The skilled artisan is aware of various methods for producing the edible hydrogel microcarriers-comprising cells.

According to a specific embodiment of the invention, there is provided a method of producing edible hydrogel microcarriers-comprising cells, the method comprising:
(a) contacting a first solution comprising a cross-linker and a second solution comprising a polysaccharide, wherein at least one of said first solution and said second solution comprises cells so as to obtain a cross linked hydrogel comprising said cells;
(b) forming the hydrogel microcarriers-comprising cells from said cross linked hydrogel, such that the microcarriers are devoid of a cell-free core.

It will be appreciated that the forming can be part of the contacting (such as in the case of alginate drops that form particles upon contacting with the cross linker).

According to a specific embodiment, at least one of the first or second solution comprises the protein as described herein.

According to a specific embodiment, the solution which comprises the polysaccharide also comprises the protein.

As mentioned, the cells can be in the first solution, second solution, or both.

Either of the solutions may comprise serum, serum replacement, growth factors and/or additives. The solutions may be xeno-free, i.e., devoid of animal contaminants (e.g., below 0.05% by weight) such as serum.

As used herein “cells” refer to non-human cells typically from an edible animal.

As used herein “animal cells” refers to any non-human cell (e.g., mammals, avian, insect, fish).

The cells can be wild-type cells or genetically modified cells (e.g., transgenic, genome edited).

According to a specific embodiment, the cells are terminally differentiated.

According to a specific embodiment, the cells are partially differentiated (e.g., blood-derived mesenchymal precursor cells, neural progenitor cells, multipotent adult progenitor cells, mesodermal progenitor cells, spinal cord progenitor cells).

According to a specific embodiment, the cells are stem cells (e.g., neural stem cells, multipotent stem cells from subventricular forebrain region, ependymal-derived neural stem cells, hematopoietic stem cells, liver-derived hematopoietic stem, marrow-derived stem cell, adipose-derived stem cells, islet-cells producing stem cells, pancreatic-derived pluripotent islet-producing stem cells, mesenchymal stem cells).

According to a specific embodiment, the cells are stem cells selected from the group consisting of mesenchymal stem cells and embryonic stem cells.

According to a specific embodiment, the cells are mesenchymal stem cells.

The phrase “embryonic stem cells” refers to embryonic cells which are capable of differentiating into cells of all three embryonic germ layers (i.e., endoderm, ectoderm and mesoderm), or remaining in an undifferentiated state. The phrase “embryonic stem cells” may comprise cells which are obtained from the embryonic tissue formed after gestation (e.g., blastocyst) before implantation of the embryo (i.e., a pre-implantation blastocyst), extended blastocyst cells (EBCs) which are obtained from a post-implantation/pre-gastrulation stage blastocyst (see WO2006/040763), embryonic germ (EG) cells which are obtained from the genital tissue of a fetus, and cells originating from an unfertilized ova which are stimulated by parthenogenesis (parthenotes).

Induced pluripotent stem cells (iPS; embryonic-like stem cells), are cells obtained by de-differentiation of adult somatic cells which are endowed with pluripotency (i.e., being capable of differentiating into the three embryonic germ cell layers, i.e., endoderm, ectoderm and mesoderm). According to some embodiments of the invention, such cells are obtained from a differentiated tissue (e.g., a somatic tissue such as skin) and undergo de-differentiation by genetic manipulation which re-program the cell to acquire embryonic stem cells characteristics.

The phrase “adult stem cells” (also called “tissue stem cells” or a stem cell from a somatic tissue) refers to any stem cell derived from a somatic tissue [of either a postnatal or prenatal animal (especially the human)]. The adult stem cell is generally thought to be a multipotent stem cell, capable of differentiation into multiple cell types. Adult stem cells can be derived from any adult, neonatal or fetal tissue such as adipose tissue, skin, kidney, liver, prostate, pancreas, intestine, bone marrow and placenta.

Hematopoietic stem cells, which may also refer to as adult tissue stem cells, include stem cells obtained from blood or bone marrow tissue of an individual at any age or from cord blood of a newborn individual.

Placental and cord blood stem cells may also be referred to as “young stem cells”.

Mesenchymal stem cells are multipotent stromal cells that can differentiate into a variety of cell types, including osteoblasts (bone cells), chondrocytes (cartilage cells), myocytes (muscle cells) and adipocytes (fat cells which give rise to marrow adipose tissue). The term encompasses multipotent cells derived from the marrow as well as other non-marrow tissues, such as placenta, umbilical cord blood, adipose tissue, adult muscle, corneal stroma or the dental pulp of deciduous baby teeth. The cells do not have the capacity to reconstitute an entire organ.

According to a specific embodiment, the cells are selected from the group consisting of erythrocytes, adipocytes, fibroblasts and muscle cells.

According to a specific embodiment, the microcarriers do not allow sufficient fusion to form cell fibers (i.e., muscle fibers).

According to a specific embodiment, the cells are of domesticated animals (e.g., duck, chicken, fish).

According to a specific embodiment, the cells are produced by in vitro expansion e.g., stem cell expansion.

According to a specific embodiment, the cells are produced by in vitro differentiation.

As used herein “partially differentiated” are cells which are committed to at least one lineage (e.g., mesodermal) and therefore do not have the potential to differentiate to all three lineages. These are also referred to as progenitor cells, e.g., adipogenic cells or myogenic cells.

As used herein “terminally differentiated cells” are cells that have undergone the process of differentiation to become specialized and acquire a specific function e.g., According to a specific embodiment adipocytes, muscle cells, blood cells, cartilage cells, bone cells, connective tissue cells, fibroblasts, cardiomyocyte, hepatocytes, cardiomyocyte, epithelial cell and/or gill cells.

According to a specific embodiment, the cells are fibroblasts or epithelial cells.

According to a specific embodiment, the cells are muscle cells or cells which can differentiate to muscle cells, also referred to as “muscle progenitor cells” or “cells of the mesoderm lineage”, e.g., myogenic cells.

As used herein, the term “myogenic cell” refers to any cell which can differentiate into a muscle cell.

The different myogenic cells may be characterized by cellular marker profiles, for example, MyoD+, Pax7 and Myf5 for myoblasts, Pax3, myosin heavy chain for muscle precursors, Prox1, Mef2ca, Prdml (E. A. Specht et al., 2023).

According to a specific embodiment, the cells are fat cells (adipocytes), cells of the mesoderm lineage” or cells which can differentiate to fat cells, adipocytes progenitor.

According to a specific embodiment, the cells are stem cells or partially differentiated cells.

According to an embodiment, the cells are terminally differentiated cells.

According to a specific embodiment, the cells are of the mesodermal lineage, though other lineages are contemplated herein (endodermal and ectodermal lineages).

In some embodiments, the cells are satellite cells, myoblasts, myocytes, adipocytes, fibroblasts, induced pluripotent stem cells, hepatocytes, vascular endothelial cells, pericytes, embryonic stem cells, mesenchymal stem cells, extraembryonic cell lines, somatic cell lines, or chondrocytes.

In some embodiments, the cells are myogenic cells. In some embodiments, the myogenic cells myogenic cells that are cultured in the cultivation system). These myogenic cells include, but are not limited to, myoblasts, myocytes, satellite cells, side population cells, muscle derived stem cells, mesenchymal stem cells, myogenic pericytes, or mesoangioblasts. In other embodiments, the myogenic cells are not natively myogenic (e.g. are non-myogenic cells that are differentiated to become myogenic cells in vitro). In some embodiments, non-myogenic cells include embryonic stem cells, induced pluripotent stem cells, extraembryonic cell lines, and somatic cells other than muscle cells.

In some embodiments, non-myogenic cells are modified to become myogenic cells through the expression of one or more myogenic transcription factors. In exemplary embodiments, the myogenic transcription factor is MYOD1, MYOG, MYF5, MYF6, PAX3, PAX7, paralogs, orthologs, or genetic variants thereof.

In some embodiments, cells are modified to extend their renewal capacity e.g., such as through inactivation of cyclin-dependent kinase inhibitor (CKI) proteins and/or activation of Telomerase reverse transcriptase (TERT). These are immortalized cells. Accordingly, in some embodiments, immortalized cells comprise a polynucleotide sequence expressing TERT. Alternatively or additionally, according to some embodiments, cells may comprise one or more loss-of-function mutations in the endogenous genes encoding CKI proteins. In some embodiments, cells comprise loss-of-function mutations in CKI proteins pi 5, pi 6, paralogs, orthologs, or genetic variants thereof. Such overexpression and inactivation strategies are described in WO2019014652, which is hereby incorporated by reference in its entirety.

In some embodiments, the cells are anchorage-dependent cells and are cultivated on a substrate. In some embodiments, the cells are anchorage independent cells.

According to some embodiments the cells are of Rainbow Trout or Gilthead Bream.

An exemplary cell is the epithelial Sea2Cell RT™ ovary cell from RT-ovary generated exclusively by the present inventors as described at from rainbow trout. See Bain et al., 2013 Aquaculture, 376-379, 59-63.

Alternatively, the cells can be isolated from an aquatic species using methods known in the art. Such a process is described in WO2020149791 which is hereby incorporated by reference in its entirety.

An exemplary embodiment is provided herewith. Isolation of primary muscle and fat cells from an aquatic species is described infra. This can be accomplished by isolating muscle and/or fat cells from various species and culturing them in vitro. For example, explants from freshly sacrificed animals are separated into small pieces. Explants are incubated in cell culture media, such as Grace's insect media, DMEM high glucose (HyClone SH30022.01, Fischer Scientific, Waltham MA), NUTRISTEM® MSC XF supplement mix (Biological Industries, Cromwell, CT), NUTRISTEM® MSC XF basal media (Biological Industries, Cromwell, CT), Myocult™ SF expansion human 1Ox (Stemcell Technologies™, Cambridge, MA) HyClone™ media for undifferentiated mesenchymal stem cells (SH30879.01, GE Fifescience, Boston, MA), Hyclone stem cell 1Ox Supplement (HyClone™ SH30878.02, Fischer Scientific, Waltham MA), Feibovitz's-15 (F-15) media (ThermoFisher, Waltham, MA), M199 media (ThermoFisher, Waltham, MA), MPS media (ThermoFisher, Waltham, MA), Pj-2 media, NCTC 135 media (ThermoFisher, Waltham, MA), MM insect media (Sigma-Aldrich®, St. Fouis, MO), or TC 100 media ((ThermoFisher, Waltham, MA), and combinations thereof. The cell culture media may additionally comprise serum, e.g., 10% fetal bovine serum (FBS) and/or 5% Penicillin/Streptomycin (PS). Fresh media is added periodically, preferably every 2-3 days. Alternatively the medium is scrum-free. Optionally, explants are periodically rinsed. The rinsing can be performed every 2-3 days until explants are removed from the culture media.

The explant/cell cultures are incubated at 18-30° C. without carbon dioxide. The identity of muscle stem cells in the population is determined by morphological identification and/or PCR amplification of muscle genes (markers of myogenic cells are described elsewhere herein). The identity of fat cells is also determined by morphological identification and/or PCR amplification of adipose-specific genes, e.g., ppary, cebpb, bmp4 and fas (E. A. Specht et al., 2023). Cultures may be tested for mycoplasma and/or other pathogens using standard techniques such as direct growth on broth/agar, specific DNA staining, PCR amplification, ELISA, RNA labeling and enzymatic procedures (e.g. enzymatic conversion of ADP to ATP, etc.), PlasmoTest™ (InvivoGen, San Diego, CA), BAM 4/LST-MUG (Feng et al. (2002) available from the fda.gov/food/laboratory-methods-food website, chapters 4 and 5), and combinations thereof. Once the cells are isolated they can be cultured till confluence of near confluence (e.g., at least 70%), 80%, 90% or more) and passaged as needed.

Cells can be cryopreserved using standard cryopreservation conditions and standard cryopreservation media (see, for example, protocols and reagents available on the internet from Thermo Fisher Scientific, Nippon Genetics, and ATCC).

Immortalization of isolated primary cells (e.g., muscle and/or fat cells) can be done as described by D. L Kaplan et al., 2023 utilizing the spontaneous immortalization crisis. Non-integrating immortalization methods are also well known in the art such as using recombinant adenoviral vectors (adm); non-integrative lentiviral vectors like lentiflash particles (vectalys), inducible lentiviral vectors (Bar-Nur et al. (2018) Stem Cell Reports 10:1505-1521), and integrase-deficient lentiviral vectors (Chick et al. (2012) Human Gene Therapy 23:1247-1257); and/or mini circles (Kim et al. (2017) Stem Cell Research 23:87-94) are used to integrate and overexpress the growth genes of interest. Adenoviral vectors include an Ad5-derived, ElA-deleted adenoviral vector expressing the full-length murine MyoD cDNA under the transcriptional control of Rous sarcoma virus LTR (Lattanzi et al. (1998) J Clin. Invest. 101(10): 2119-2128); Ad-MyoD, Ad-m-MYOD1 (Vector Biolabs, Cat. No 1492, ADV-265351); human type-5 adenovirus (Suchiro et al. (2010) FEBS Letters 584:3545-3549) and white sturgeon adenovirus (WSAdV-1 (Hendrick et al. (1985) Can J Fish Aquat Sci 42:1321-1325; Hendrick et al. (1990) Dis Aquat Org 8:39-44). An example of non-integrative lentiviral vectors is commercially available lentiflash particles (Vectalys); this includes the customizable pRLP-MS2 plasmid (Vectalys, Toulouse, France), where a muscle growth gene, as mentioned above, is cloned into the vector and is transduced along with pRLP-MCP and VSVG plasmids. Alternatively, inducible lentiviral vectors like tetOP-MyoD and M2rtTA are co-expressed in the isolated muscle cells (Bar-Nur et al. (2018)). Similarly, suitable integrase-deficient lentiviral plasmids can be constructed according to Chick et al. (2012), including a combination of pHR′ SIN-cPPT-SFFV-cGFP-WPRE, pHR′ SIN-cPPT-SFFV-NogoB-WPRE, and pCMV delta R8.74 D64V plasmids. Finally, DNA minicircles expressing hPax7 are cloned according to Kim et al. (2017).

Otherwise, chemical immortalization of cells is stimulated by use of telomerase activators such as Cycloastragenol (Sigma, SMB00372-20 MG; Fauce et al. (2008) J Immunol. 181 (10): 7400-7406), Genistein from Glycine Max (soybean) (Sigma, G6776-5 MG; Chau et al. (2007) Carcinogenesis 28 (11): 2282-2290), or Resveratrol (Sigma, R5010-100 MG; Xia et al. (2008) British Journal of Pharmacology 155:387-394; Zhai et al. (2016) Oncology Letters 11:3015-3018). Isolated primary cells at about 70% confluence are used for transduction. The immortality of primary cells is verified by PCR expression of muscle stem cell and/or growth genes or by the ability of cells to be passaged beyond 10 times. Telomerase activity is assessed by telomerase repeated amplification ELISA.

Alternatively contemplated herein is generating iPS cells from isolated cells. According to a specific embodiment, primary muscle and/or fat cells, and/or immortalized muscle and/or fat cells are reprogrammed by transfection with episomal plasmids (see, for example, Chandrobose et al. (2018) Stem Cell Research & Therapy 9:68; Slamecka et al. (2016) Cell Cycle 15 (2): 234-249) or by integration- and xeno-free mRNA transfection (Lec et al. (2016) Stem Cells International 2016:6853081).

Episomal plasmids from the Yamanaka cocktail are used: pCXLEhOct3/4-shp53-F, pCXLE-hSK, pCXLE-hUL, and pCXLE-EGFP (Addgene, Watertown, MA) as well as the Yamanaka cocktail of Oct4, Sox2, Klf4 and Myc (Takahashi et al. (2007) Cell 131:861-872; Okita et al. (2011) Nat Methods 8:409-412; Rosello et al. (2013) Elife 2: e00036).

Immediately following transfection, cells are placed in culture media. Appropriate culture media include Grace's Insect Media supplemented with 10% FBS and 2% PS (Ma et al., 2017; George and Dhar, 2010) and/or mesenchymal stem cell media. Media is changed daily and on day 7, cells are seeded onto MATRIGEI® (Corning, Tewksbury, MA) for feeder-free induced pluripotent stem (iPS) cell derivation. Subsequently, the media is changed to mTeSR1 (STEMCELL TECHNOLOGIES™, Cambridge, MA), preferably supplemented with sodium butyrate. Media is then preferably supplemented at a later date with small molecules SMC4 cocktail (containing small molecules: PD0325901, CHIR99021, Thiazovivin, and SB431542 (FOCUS Biomolecules, Plymouth meeting, PA)) until initial colonies are formed.

Alternatively or additionally provided herein is generation of differentiated cells e.g., muscle, fat cells, cardiomyocytes and hepatocytes from pluripotent embryonic stem cells.

In other embodiments the muscle and/or fat cells are derived from pluripotent embryonic stem cells, such as cells from the blastocyst stage and fertilized eggs.

Pluripotent embryonic stem cells are initially cultured essentially as described above. Muscle cells are differentiated from embryonic stem cells or induced pluripotent stem cells as described in Salani et al. (J Cell Mol Med (2012) 16(7): 1353-1364) and Chai and Pourquie (Development (2017) 144:2104-2122). Adipocytes are differentiated from embryonic stem cells and induced pluripotent stem cells as described in Barberi et al. (PLOS Med (2005) 2(6): el61), Mohsen-Kanson et al. (Stem Cells (2014) 32:1459-1467) and Hafner et al. (Scientific Reports (2016) 6: Article Number 32490).

Exemplary protocols for differentiating cells in culture are provided in D. L Kaplan et al., 2023, E. Xu et al., 2023.

According to a specific embodiment, the cells are of an aquatic species.

According to a specific embodiment, the cells are fish cells.

According to a specific embodiment, the fish cells are of a fish species selected from a group comprising of salmon, grouper, tuna, snapper, mackerel, cod, trout, carp, catfish, shark and sardine.

According to a specific embodiment, the cells are of an avian or a mammalian species.

According to a specific embodiment, the cells are stem cells.

According to a specific embodiment, the cells are myogenic cells.

According to a specific embodiment, the cells are adipocytes, muscle cells, blood cells, cartilage cells, bone cells, connective tissue cells, fibroblasts, cardiomyocyte, hepatocytes, cardiomyocyte, epithelial cell and/or gill cells.

As used herein “aquatic species” refers to isolated cells of fish or crustaceous. Typically the aquatic species is of an edible (e.g., by human) species. According to some embodiments, the aquatic species is a farmed fish or crustaceans (e.g. shrimp or prawns), and in particular, any one of the group consisting of Shrimp, Prawns, Crabs, Lobsters and Crayfishes. In some specific embodiments the farmed crustacean is a shrimp or prawn. Suitable shrimps or prawns can be selected from the group consisting of Litopanacus vannamei, Panacus monodon, Penacus japonicas and Macrobrachium rosenbergii.

The term “farmed crustacean” or “farmed fish” as used herein refers to a fish or crustacean which is either strictly or partially aquatic (i.e. living at least a portion of the organism's life cycle in water), and which is cultivated (i.e. grown) by man, in an aquaculture environment.

Finfish (e.g. flounder and whiting), marine crustaceans (e.g. prawns, shrimp, lobsters and crabs) and marine mollusks (e.g. oysters and abalone) can be cultured in seawater. Fresh water aquaculture is suitable for fresh water species, including fish (e.g. tilapia, trout), crustaceans (e.g. crayfish) and fresh water mollusks (e.g. clams). Some species are particularly suited for culture in brackish water (carp, catfish).

According to a specific embodiment, the aquatic animal is a marine fish or crustacean.

According to a specific embodiment, the aquatic species is a diadromous fish or crustacean.

According to a specific embodiment, the aquatic species is a freshwater fish or crustacean.

According to a specific embodiment, the aquatic species is carnivore (e.g., carnivore fish).

According to a specific embodiment, the aquatic species is herbivore (e.g., herbivore fish).

According to a specific embodiment, the aquatic species is omnivore (e.g., omnivore fish).

According to a specific embodiment, the fish is a finfish.

According to a specific embodiment, the fish are fish of the salmonid group, for example, cherry salmon (Oncorhynchus masou), Chinook salmon (Oncorhynchus tshawytscha), chum salmon (Oncorhynchus keta), coho salmon (Oncorhynchus kisutch), pink salmon (Oncorhynchus gorbuscha), sockeye salmon (Oncorhynchus nerka) and Atlantic salmon (Salmo salar). Other fish of interest for aquaculture include, but are not limited to, various trout, as well as whitefish such as tilapia (including various species of Oreochromis, Sarotherodon, and Tilapia), grouper (subfamily Epinephelinae), sea bass, sea bream, catfish (order Siluriformes), bigeye tuna (Thunnus obesus), carp (family Cyprimidae) and cod (genus Gadus). Other fish species that may be used according to the present teachings are provided hereinbelow (Table 2).

TABLE 2
List of animal species used in aquaculture

| Common | Scientific | Common | Scientific | Common | Scientific |
|---|---|---|---|---|---|
| name | name | name | name | name | name |
| Siberian | Acipen serbaeri | Paco | Piaractus | Bigmouth | Ictiobus |
| sturgeon | | | mesopotamicus | buffalo | cyprinellus |
| Sterlet | Acipenser ruthenus | Black | Ictalurus | Bocachico | Ichthyoelephas |
| sturgeon | | bullhead | melas | | humeralis |
| Starry | Acipenser | Channel | Ictalurus | Bocachico | Prochilodus |
| sturgeon | stellatus | catfish | punctatus | | reticulatus |
| White | Acipenser | Bagrid | Chrysichthys | Dorada | Brycon moorei |
| sturgeon | transmontanus | catfish | nigrodigitatus | | |
| Beluga | Huso | Wels | Siluris | Cachama | Colossoma |
| | | catfish | glanis | | macropomum |
| Arapaima | Arapaima gigas | Pangas | Pangasius | Cachama | Piaractus |
| | | catfish | | blanca | brachypomus |
| African | Heterotis | Striped | Pangasius | Striped bass | Morone |
| bonytongue | niloticus | catfish | sutchi | | saxatilis |
| European | Anguilla | Mudfish | Clarias | European | Dicentrarchus |
| eel | | | anguillaris | seabass | labrax |
| Japanese | Anguilla japonica | Philippine | Clarias | Hong Kong | Epinephelus |
| eel | | catfish | batrachus | grouper | akaara |
| American | Anguilla rostrata | Hong Kong | Clarias | Areolate | Epinephelus |
| eel | | catfish | fuscus | grouper | areolatus |
| Milkfish | Chanos | North | Clarias | Greasy | Epinephelus |
| | | African | gariepinus | grouper | tauvina |
| | | catfish | | | |
| Freshwater | Abramis brama | Bighead | Clarias | Spotted | Plectropomus |
| bream | | catfish | macrocephalus | coralgrouper | maculatus |
| Asp | Aspius | African | Heterobranchus | Silver perch | Bidyanus |
| | | catfish | bidorsalis | | |
| Catla | Catla | Sampa | Heterobranchus | Large mouth | Micropterus |
| | | | longifilis | black bass | salmoides |
| Goldfish | Carassius auratus | South | Rhamdia sapo | European | Perca |
| | | American | | perch | fluviatilis |
| | | catfish | | | |
| Crucian | Carassius | Atipa | Hoplosternum | Pike-perch | Stizostedion |
| carp | | | littorale | | lucioperca |
| Mud carp | Cirrhinus | Northern | Esox lucius | Bluefish | Pomatomus |
| | molitorella | pike | | | saltatrix |
| Mrigal | Cirrhinus mrigala | Ayu | Plecoglossus | Greater | Seriola |
| carp | | sweetfish | altivelis | amberjack | dumerili |
| Grass | Ctenopharyn | Vendace | Coregonus | Japanese | Seriola |
| carp | godonidellus | | albula | amberjack | quinqueradiata |
| Common | Cyprinus carpio | Whitefish | Coregonus | Snubnose | Trachinotus |
| carp | | | lavaretus | pompano | blochii |
| Silver | Hypophthalmichthys | Pink salmon | Oncorhynchus | Florida | Trachinotus |
| carp | molitrix | | gorbuscha | pompano | carolinus |
| Bighead | Hypophthalmichthys | Chum salmon | Oncorhynchus | Palometa | Trachinotus |
| carp | nobilis | | keta | pompano | goodei |
| Orangefin | Labeo calbasu | Coho salmon | Oncorhynchus | Japanese | Trachurus |
| labeo | | | kisutch | jack | japonicus |
| | | | | mackerel | |
| Roho | Labeo rohita | Masu salmon | Oncorhynchus | Mangrove | Lutjanus |
| labeo | | | masou | red | argentimaculatus |
| | | | | snapper | |
| Hoven's | Leptobarbus | Rainbow | Oncorhynchus | Yellowtail | Ocyurus |
| carp | hoeveni | trout | mykiss | snapper | chrysurus |
| Wuchang | Megalobrama | Sock eye | Oncorhynchus | Dark | Acanthopagrus |
| bream | amblycephala | salmon | nerka | seabream | schlegeli |
| Black | Mylopharyngodon | Chinook | Oncorhynchus | White | Diplodus sargus |
| carp | piceus | salmon | tshawytscha | seabream | |
| Golden | Notemigonus | Atlantic | Salmo salar | Crimson | Evynnis japonica |
| shiner | crysoleucas | salmon | | seabream | |
| Nilem | Osteochilus | Sea trout | Salmo trutta | Red | Pagrus major |
| carp | hasselti | | | seabream | |
| White | Parabramis | Arctic char | Salvelinus | Red porgy | Pagrus |
| amur bream | pekinensis | | alpinus | | |
| Thai | Puntius | Brook trout | Salvelinus | Goldlined | Rhabdosargus |
| silver barb | gonionotus | | fontinalis | seabream | sarba |

| Common | Scientific | Common | Scientific |
|---|---|---|---|
| name | name | name | name |
| Common | Centropomus | Java barb | Puntius |
| snook | undecimalis | | javanicus |
| Barramundi | Lates | Roach | Rutilus |
| | calcarifer | | |
| Nile perch | Lates | Tench | Tinca |
| | niloticus | | |
| Murray cod | Maccullochella | Pond loach | Misgurnus |
| | peeli | | anguillicaudatus |
| Golden perch | Macquaria | Climbing | Anabas |
| | ambigua | perch | testudineus |
| Gilthead | Sparus | Snakehead | Channa argus |
| seabream | aurata | | |
| Red drum | Sciaenops | Turbot | Psetta maxima |
| | ocellatus | | |
| Green terror | Aequidens | Lake trout | Salvelinus |
| | rivulatus | | namaycush |
| Blackbelt | Cichlasoma | Atlantic | Gadus |
| cichlid | maculicauda | cod | morhua |
| Jaguar | Cichlasoma | Pejerrey | Odontesthes |
| guapote | managuense | | bonariensis |
| Mexican | Cichlasoma | Lai | Monopterus |
| mojarra | urophthalmus | | albus |
| Pearlspot | Etroplus | Snakeskin | Trichogaster |
| | suratensis | gourami | pectoralis |
| Three spotted | Oreochromis | Indonesian | Channa |
| tilapia | andersonii | snakehead | micropeltes |
| Blue | Oreochromis | Bastard | Paralichthys |
| tilapia | aureus | halibut | olivaceus |
| Longfin | Oreochromis | Goldlined | Siganus |
| tilapia | macrochir | spinefoot | guttatus |
| Mozambique | Oreochromis | Marbled | Siganus |
| tilapia | mossambicus | spinefoot | rivulatus |
| Nile tilapia | Oreochromis | Southern | Thunnus |
| | niloticus | bluefin tuna | maccoyii |
| Tilapia | Oreochromis | Northern | Thunnus |
| | spilurus | bluefin tuna | thynnus |
| Wami tilapia | Oreochromis | Kissing | Helostoma |
| | urolepis | gourami | temmincki |
| Blackchin | Sarotherodon | Spotted | Channa |
| tilapia | melanotheron | snakehead | punctatus |
| Tilapia | Tilapia | Common sole | Solea |
| | guineensis | | vulgaris |
| Redbreast | Tilapia | Lebranche | Mugil liza |
| tilapia | rendalli | mullet | |
| Redbelly | Tilapia | Pacific fat | Dormitator |
| tilapia | zillii | sleeper | latifrons |
| Golden | Liza aurata | Marble goby | Oxyeleotris |
| grey mullet | | | marmorata |
| Largescale | Liza | White-spotted | Siganus |
| mullet | macrolepis | spinefoot | canaliculatus |
| Gold-spot | Liza parsia | Giant gourami | Osphronemus |
| mullet | | | goramy |
| Thinlip | Liza ramada | Striped | Channa |
| grey mullet | | snakehead | striata |
| Leaping | Liza saliens | | |
| mullet | | | |
| Tade mullet | Liza tade | | |
| Flathead | Mugil cephalus | | |
| grey mullet | | | |
| White | Mugil curema | | |
| mullet | | | |

Source: FAO corporate document repository, List of animal species used in aquaculture

Contemplated are both lower-value staple food fish species [e.g., freshwater fish such as carp, tilapia and catfish] and higher-value cash crop species for luxury or niche markets [e.g., mainly marine and diadromous species such as shrimp, salmon, trout, yellowtail, seabass, seabream and grouper]).

According to a specific embodiment, the cells are of Gilthead seabream (GHB), rainbow trout (e.g., epithelial cells originated from the ovary or Steelhead Trout.

According to a specific embodiment, the cells are of one cell type.

According to a specific embodiment, the cells are of two or more (e.g., 3, 4) cell types.

According to a specific embodiment, the cells comprise one or two cell types.

As mentioned, any of the solutions (and optionally the composition of the hydrogel) may comprise an additive selected from the group consisting of one or more of color enhancers, taste enhancers, nutritional additives, fiber, carbohydrate, meal, preservatives, fats and/or oils.

For example, a composition which comprises a cell species or cell type is colored with a specific color (e.g., fish cells-blue; bovine cells-red; likewise, adipocyte-yellow, and myocytes-red, as non-limiting examples).

According to a specific embodiment, the additive is selected from the group consisting of an Omega 3, Omega 6, Fatty acid, Vitamin D, Vitamin A, Taurine, amino acid, dietary fiber and an anti oxidant.

As mentioned, one of the solutions comprises a cross linker.

According to some embodiments, the cross-linker is a chemical cross-linker.

According to other or additional embodiments, the cross-linker is an enzymatic cross-linker.

The type of cross-linker typically changes with, or dependent on the identity of the polymer.

According to some embodiments, the polymer is alginate and the cross-linker is Ca⁺².

According to some embodiments, the polysaccharide and the protein are cross linked to one another, and/or each is cross-linked independently.

According to some embodiments, the particles comprise the cross-linking agent (e.g., Ca⁺², an enzyme).

Chemical cross-linking of proteins can be achieved by organic acid treatment, e.g., citric acid. Examples of other acids which can be used in cross-linking the protein portion include but are not limited to, tannic, malic, lactic and hydrochloric acid.

U.S. Pat. No. 9,752,122 describes a specific embodiment of polysaccharide (pectin) and protein chemical cross-linking e.g., creating pectin-thiopropionylamide (PTP) by derivatization of pectin with cystamine; thiolating a cardosin polypeptide: cross-linking the PTP and thiolated cardosin to form a hydrogel. Alternatively or additionally, cross-linking through oxidative disulfide bond formation. In this example, polysaccharide and/optionally protein may be cross-linked under mild conditions using (the oxidized form of) glutathione disulfide (GSSG) obtained by bubbling air into a solution of high-grade glutathione (GSH, e.g., such as health-food store grade glutathione).

As used herein “cross-linking enzyme” is an enzyme that catalyzes the formation of covalent bonds between one or more polypeptides.

Exemplary cross-linking enzymes are selected from the group consisting of transglutaminase, sortase, subtilisin, tyrosinase, laccase, peroxidase, and lysyl oxidase. As used herein, “transglutaminase” or “TG” refers to an enzyme (R-glutamyl-peptide amine glutamyl transferase) that catalyzes the formation of a peptide (amide) bond between gamma.-carboxyamide groups and various primary amines, classified as EC 2,3.2,13. Transglutaminases catalyze the formation of covalent bonds between polypeptides, thereby cross-linked polypeptides. Cross-linking enzymes such as transglutaminase are used in the food industry to improve texture of some food products such as dairy, meat and cereal products. It can be isolated from a bacterial source, a fungus, a mold, a fish, a mammal, or a plant. According to a specific embodiment, the TG concentration is 20-200 units/gr protein.

According to an embodiment, cross-linked particles are formed in solution, such as by using microfluidics.

According to an embodiment, cross-linked particles are formed in a mold, so as to provide a desired size and shape of the formed particles. Molding can be done using a 3D-printed mold having a desired geometry and pattern.

Besides, or in addition to, cross-linking or gel-formation, the microparticles can be formed by any other methodology or technique known in the art for particles formation.

In some embodiments, microparticles (interchanged with microcarriers or particles) are produced by spraying (e.g., electrospraying) an aqueous solution into a water-miscible organic solvent (e.g., an alcoholic solvent or solution). Measures are taken to use techniques which are compatible with cell viability.

Alternatively, microparticles can be produced by invention emulsion techniques, solvent-removal technique, molding, and any other methods known in the art, preferably such methods that can be successfully executed using GRAS or food contact substances.

According to a specific embodiment, forming the microcarriers is by microfluidics.

According to an embodiment, the edible microcarriers may be prepared by forming the components, polysaccharide and polypeptide, into a cross-linked hydrogel, lyophilization of cross-linked hydrogel, and shaping (e.g., cutting) the lyophilized gel into appropriate sizes.

According to a specific embodiment, the microcarriers may be generated by a coaxial bead generator such as the Nisco coaxial airflow bead generator.

According to a specific embodiment, forming the microcarriers is by 3D printing.

According to a specific embodiment, contacting is concomitantly with said forming.

According to a specific embodiment, contacting is prior to forming. For example a step of lyophilization can be done between contacting and forming.

The cells in the particles display at least 20%, 30%, 40%, 50%, 60%, 70%, 80%, 90% viability, as can be determined by Calcein AM and Ethidium Bromide.

The cells may be subjected to culturing, such as for cloning cells, expanding cells and/or subjecting the cells to differentiation protocols as desired.

Thus, according to an aspect of the invention there is provided a method of producing a cell line, the method comprising passaging in a culture medium the microcarriers as described herein wherein the microacarriers comprise heterogeneous cells to obtain a cell line.

Alternatively or additionally, there is provided a method of culturing cells, the method comprising incubating the composition which comprises the microcarriers and cells in culture under conditions which allow proliferation and/or differentiation of said cells, thereby culturing the cells.

The cells may remain as single cells in the particles, or may aggregate to form clusters or clumps or even form spheroids in the microcarriers.

The term “spheroid” refers to a synthetic three-dimensional aggregation of cells that closely resembles a small, rounded mass or sphere. Unlike traditional two-dimensional cell cultures where cells grow on a flat surface, spheroids allow cells to interact with each other in a more physiologically relevant manner, forming complex cell-cell and cell-matrix interactions. The level of differentiation of the cells in the spheroid may vary. Spheroids made of pluripotent stem cells (e.g., iPS cells or embryonic stem cells) may comprise cells of all 3 germ layers, as in an embryoid body.

Thus, according to an embodiment of the invention the cells form a microsphere (also referred to as “a spheroid”) in the microaarrier. According to a specific embodiment, the cells in the spheroid ate 10-200 cells, e.g., 50-200 cells. A plurality of spheroids can occupy the particle.

To produce large amounts of cells the use of bioreactors is contemplated.

As used herein “bioreactor” refers to a vessel, device or system designed to grow cells. Such bioreactors are described by Popovic et al. Biotechnology—Bioreactoes and Cultivation Systems for Cell and Tissue Culture—M. K. Popovic, Ralf Portner Encyclopedia of Life Support Systems (EOLSS) and further hereinbelow and in the Examples section which follows.

Selection of culture apparatus for production is based on the scale. Large-scale production preferably involves the use of dedicated devices. Continuous cell culture systems are reviewed in Furey (2000) Genetic Eng. News 20:10. Suitable bioreactors which can be used according to the present teachings include, but are not limited to, packed-bed bioreactors, fluidized-bed bioreactors, simulated microgravity bioreactors such as high aspect ratio vessel bioreactors, and slow turning lateral vessel bioreactors.

Typical bioreactors utilize a chamber filled with media, such as DMEM supplemented with serum/serum-replacement and/or growth factors and vented to ensure that there is a zero-head space in the reactor chamber. In some embodiments, the reactor chamber is then incubated at 37° C. and the media pumped through a media gas exchange module having its gas exchange tubing filled with a gas mixture as known to those of skills in the art.

In some embodiments, the bioreactor cell culture system is scalable for commercial production of viable cells. In some embodiments, the bioreactors of the present invention have been optimized for the expansion of stem cells or more differentiated cells.

According to a specific embodiment, the culturing is a suspension culture.

In other embodiments, the microcarriers are allowed to attach to a surface, such as a scaffold.

Typically, the carriers (microcarriers) are introduced into the bioreactor chamber through a sampling port. Since there is no need to seed the carriers (the cells are already comprised therein), culturing may be immediately effected, a process which typically involves medium flow/agitation/stirring.

In some embodiments, the media used for culturing can comprise a basal salt nutrient solution. A basal salt nutrient solution refers to a mixture of salts that provide cells with water and certain bulk inorganic ions essential for normal cell metabolism, maintain intra- and extra-cellular osmotic balance, provide a carbohydrate as an energy source, and provide a buffering system to maintain the medium within the physiological pH range. For example, basal salt nutrient solutions may include, but are not limited to, Dulbecco's Modified Eagle's Medium (DMEM), Minimal Essential Medium (MEM), Basal Medium Eagle (BME), RPM1 1640, Hams F-10, Ham's F-12, (beta-Minimal Essential Medium (beta-MEM), Glasgow's Minimal Essential Medium (G-MEM), and Iscove's Modified Dulbecco's Medium, and mixtures thereof. According to other embodiments the basal medium includes the ingredient of L-15 medium, as an exemplary medium which negates the need for CO₂, supplementation of essential value for marine species, e.g., fish that require growth temperatures below 37° C.

According to a specific embodiment, the culturing is performed in the presence of exogenously added growth factor.

According to a specific embodiment, the culturing is performed in the absence of exogenously added growth factor.

Presence or absence of the growth factor (or any other additive) can be in the microcarriers, in the culturing medium, or in both.

According to a specific embodiment, passaging can take place every 2-7 days, for at least 2 and optionally up to 100 passages (e.g., 5-100, 5-50, 5-30, 5-20, 5-10, 10-100, 10-50, 10-30, 10-20, 20-100, 2-50, 20-30).

According to a specific embodiment, the cells in the microcarriers form filopodia. This suggests, that the cells are able to migrate, attesting to their healthy physiological condition and ability to migrate such as to surfaces in the culturing container (such as a scaffold) or communicate with each other.

Since the particles are edible there is no need to dissociate the cells from the particles following culturing. The particles can be further coated with any substance of interest, such as the additives described herein (e.g., flavors, colorant, a protein, a polysaccharide (that can be distinct from the hydrogel's core) and the like).

Regardless of the method employed, once the particles are at hand they can be used in the food/feed/beverage industry to produce comestible products, e.g., meat (e.g., fish), or meat-like products.

Thus, the edible particles can be formed and/or combined with other molecules/substances with high nutritional value or providing improved texture or adding flavor to the final product. The other substances with which the microcarriers are combined with may be synthetic, plant-based or animal-derived or combination of same. They can also be used for the simultaneous culturing of several cell types with relevance to meat, thus allowing for the engineering of novel foods. For example, bovine and fish cells could be cultured simultaneously.

Thus, according to an aspect there is provided a food, feed or beverage comprising microcarriers (which comprise the cells) as described herein.

Also provided is a method of producing food, the method comprising fabricating food comprising the composite or composition as described herein.

According to some embodiments, the fabricating comprises molding, extruding, electrospinning and/or printing.

According to other embodiments, the particles are combined with other food ingredients to form consumables such as minced meat, burger, nugget, sausage or patty.

According to a specific embodiment, the resultant comestible composition is selected from a group comprising a cake, a ball, a burger, a canned composition, an imitation of a steak, a stick, a ground meat, a nugget and a sausage.

According to a specific embodiment, the composition is processed to impart an organoleptic sensation and texture of meat.

According to a specific embodiment, the resultant comestible composition comprises other comestible additives.

According to a specific embodiment, the resultant comestible composition comprises plant and/or animal-originated food stuffs and/or plant and/or animal-based proteins.

According to a specific embodiment, the resultant comestible composition further comprises plant-based protein (not in the microcarrier).

It is thus within the scope of the invention wherein the food, feed or beverage additionally comprises acidity regulators, anticaking agents, antifoaming agents, natural and other antioxidants, bulking agents, food coloring agents, color retention agents, emulsifiers, flavors, flavor enhancers, flour treatment agents, glazing agents, humectants, tracer gas, preservatives, probiotic microorganisms, stabilizers, sweeteners, thickeners and any mixtures thereof.

In other embodiment of the present invention, the food, feed or beverage product is a consumable, edible item having the final organoleptic properties of a meat product, and especially product(s) selected from the group consisting of a marine species (as described above), beef, lamb, duck, chicken and the like such as heart, liver, beef tongue, bone soup from allowable meats, buffalo, bison, calf liver, caribou, goat, ham, horse, kangaroo, lamb, marrow soup, moose, mutton, opossum, organ meats, pork, bacon, rabbit, snake, squirrel, sweetbreads, tripe, turtle, veal, venison, chicken, chicken liver, cornish game hen, duck, duck liver, emu, gizzards, goose, goose liver, grouse, guinea hen, liver, ostrich, partridge, pheasant, quail, squab, and turkey.

As used herein the term “about” refers to +10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween. As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

### Materials and Method

### Cell Culture

Steelhead Trout Embryo-137 (STE-137) or RT cells were cultured in an L-15 (GIBCO) medium supplemented with heat-inactivated 10% fetal bovine serum (FBS), 100 iu/ml penicillin and 100 mg/ml streptomycin on plastic cell culture petri dishes at 18° C., in an incubator. Cells were harvested from the dishes using trypsin EDTA solution B (Biological Industries), 2 ml for 5 min. Centrifugation of the cells was performed at 300 g for 5 min at 18° C. to obtain the STE-137 pellet for the encapsulation experiments. After their encapsulation as detailed below, the STE-137 microcarriers were cultured for up to 40 days. Culture medium was replaced every 3 days. STE-137 cell-seeded microcarriers were cultured in L-15 medium supplemented with 10% FBS and 1% penicillin/streptomycin. For GHB, the same protocol was applied except the cells grew in 20% serum and 22° C.

### Cell-Seeded Microcarriers Fabrication

Alginate-gelatin capsules (AGG) were fabricated using a microfluidic system. A gelatin solution (10% w/v) was prepared by dissolving 10 g of gelatin in 100 ml deionized water. The alginate solution (2%, w/v) was prepared by dissolving 2 g of sodium alginate in 100 ml of phosphate-buffered saline (PBS). Different percentages of gelatin (0%, 1%, 2%, 3%, and 4%) were added to a final mixture of 0.8% sodium alginate. The mixture was then gently heated to 37° C. and stirred until fully dissolved and then cooled to 18° C. Next, the STE-137 cells were trypsinized and suspended in the mixture at a concentration of 1×10⁶ cells/ml. The cell-containing mixture was then gently added dropwise into a calcium chloride solution (0.2M) using a syringe with a 30-gauge needle to create microcarriers with a diameter range between 0.5 to 1.5 mm. Microcarriers were washed with sterile phosphate-buffered saline (PBS) three times to remove excess calcium chloride. The encapsulated cells were then observed under a microscope to assess their viability and morphology.

### Imaging and Analysis

Encapsulated cells were imaged using a brightfield microscope at days 3, 7 and 14 after encapsulation. Images were analyzed using ImageJ software to measure the size of the capsules and the presence of filopodia.

### 3D-Adapted Cell Line

STE-137 cell-seeded microcarriers were cultured in L-15 medium supplemented with 10% FBS and 1% penicillin/streptomycin. Every 3 days, the capsules were washed with PBS and transferred into a new petri dish coated with 0.2% gelatin. The migrated cells were then cultured for an additional 7 days to form a novel 3D-adapted cell line. zz

### Fluorescence Staining

Phalloidin and DAPI fluorescence staining was performed to visualize the actin cytoskeleton and nuclei of the fish cells encapsulated in the polysaccharide matrix. The encapsulated cells were fixed in 4% paraformaldehyde for 30 minutes at room temperature and then washed with phosphate-buffered saline (PBS) three times for 5 minutes each. The fixed cells were then permeabilized with 0.1% Triton X-100 in PBS for 10 minutes and washed with PBS three times for 5 minutes each. To stain the actin cytoskeleton, the cells were incubated with 100 nM phalloidin conjugated to Alexa Fluor 594 (Thermo Fisher Scientific) for 20 minutes at room temperature. To stain the nuclei, the cells were incubated with 1 μg/ml DAPI (4′,6-diamidino-2-phenylindole) for 10 minutes at room temperature. The cells were then washed with PBS three times to remove any unbound staining reagents. The stained cells were visualized using a fluorescence microscope.

### Example 1

### Cell-Seeded Alginate-Gelatin (AGG) Microcarriers

Different percentages of gelatin (0%, 1%, 2%, 3%, and 4%) were added to a final mixture of 0.8% (w/v) sodium alginate. To determine the optimal concentration of gelatin for cell encapsulation, cell growth and migration were compared among the different gelatin concentrations. Based on the results, the optimal concentration of gelatin for cell encapsulation was found to be 2%. Therefore, this concentration was used in subsequent experiments to encapsulate the STE-137 cells in the polysaccharide-gelatin mixture.

### Example 2

### Cell Branching and Filopodia Formation

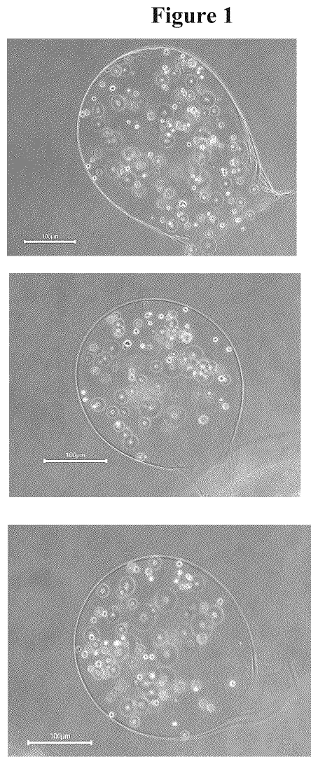

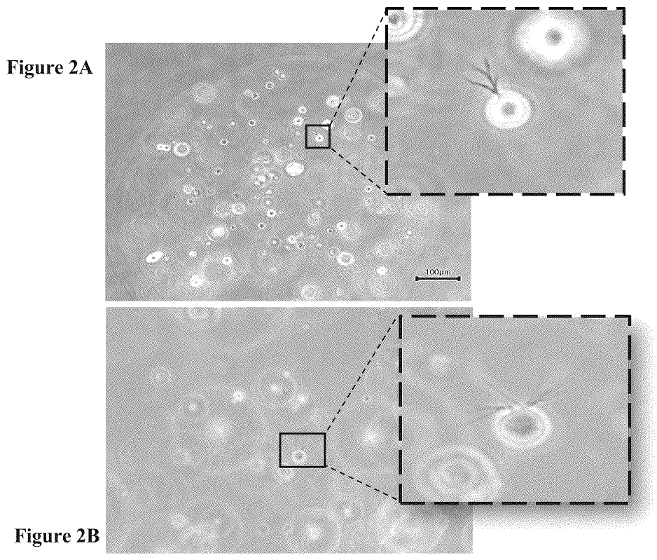

STE-137 cell line, derived from Steelhead Trout Embryos, was encapsulated in a 3D polysaccharide matrix with a final concentration of 0.8% sodium alginate and 2% of gelatin. After six days of encapsulation, the cells were observed to be attaching and spreading out within the matrix [FIG. 1]. Interestingly, the presence of filopodia was also observed on the surface of some of the cells. The presence of filopodia suggests that the cells are actively interacting with the matrix and beginning to organize themselves into more complex tissue structures (FIGS. 2A-B).

### Example 3

### 3D-Adapted STE-137 Cell Line

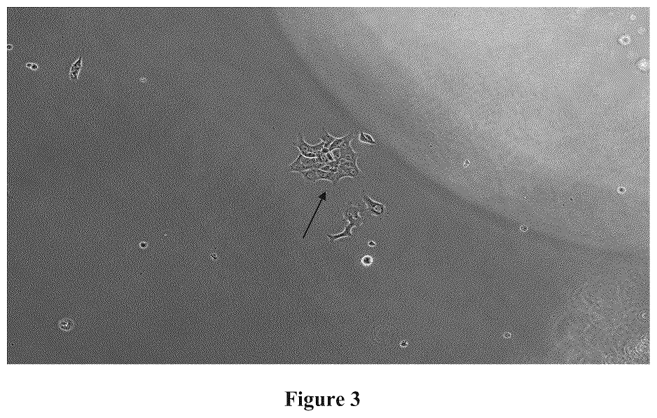

This study aimed at exploring the efficacy of AGG microcarriers as a promising platform for facilitating cell migration and proliferation. To monitor the formation of new cell lines and to track their growth over time, the cell-seeded microcarriers were transferred into fresh cell culture petri dishes every three days. This transfer allowed observing the emergence of new 3D-adapted cell lines that had developed within the AGG microcarriers (FIG. 3). These newly formed cell lines were then cultured for further analysis to explore their potential for tissue engineering applications.

### Example 4

### Actin Formation

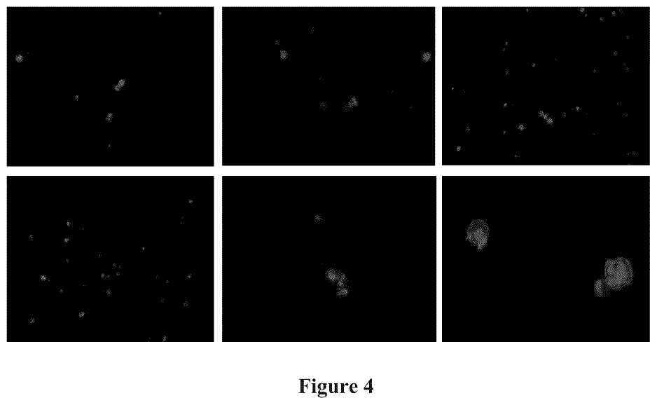

To evaluate actin formation in the STE-137 cells encapsulated in the 3D polysaccharide matrix, the cells were stained with phalloidin, a fluorescent dye that binds specifically to F-actin. The staining was performed on days 7 and 14 of encapsulation. The results showed a clear and consistent growth of actin filaments in the encapsulated STE-137 cells. The filaments were observed as thin, elongated structures with bright fluorescence signal. This observation is consistent with previous studies that have demonstrated the crucial role of actin filaments in the formation and maintenance of 3D tissue structures. These findings suggest that the 3D polysaccharide matrix is capable of supporting the growth and migration of the encapsulated STE-137 cells. Results are shown in FIG. 4.

### Example 5

### Spheroid Formation

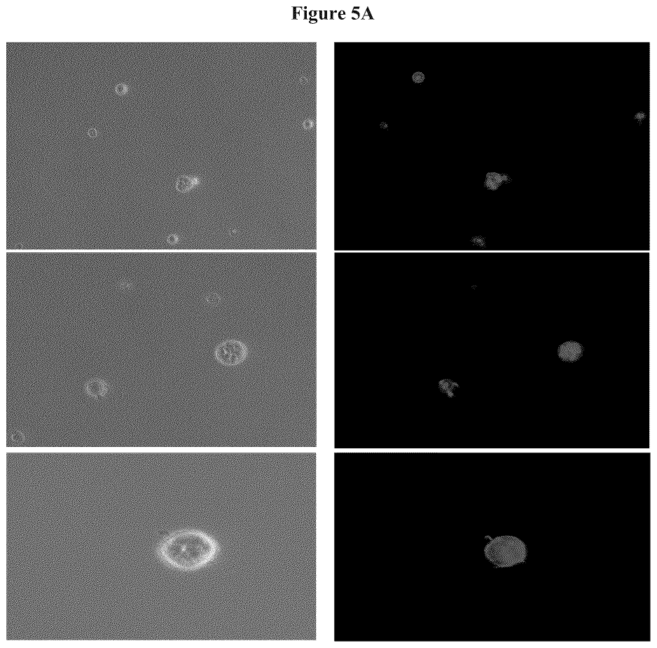

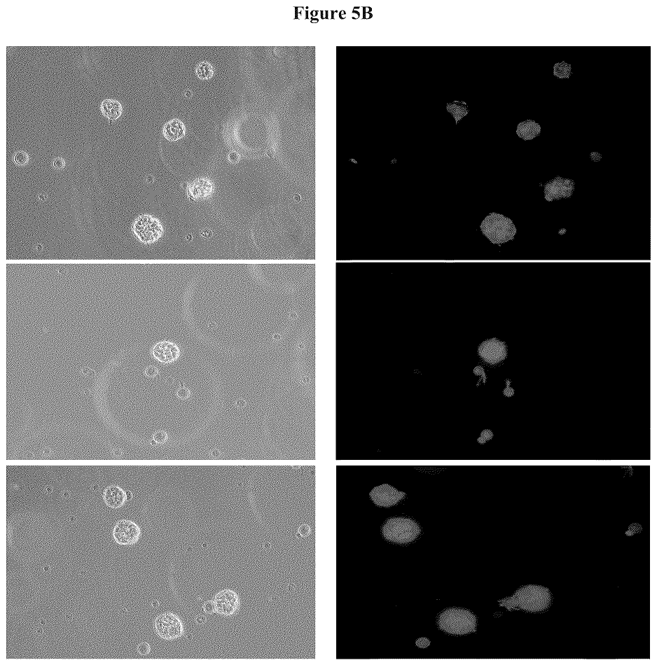

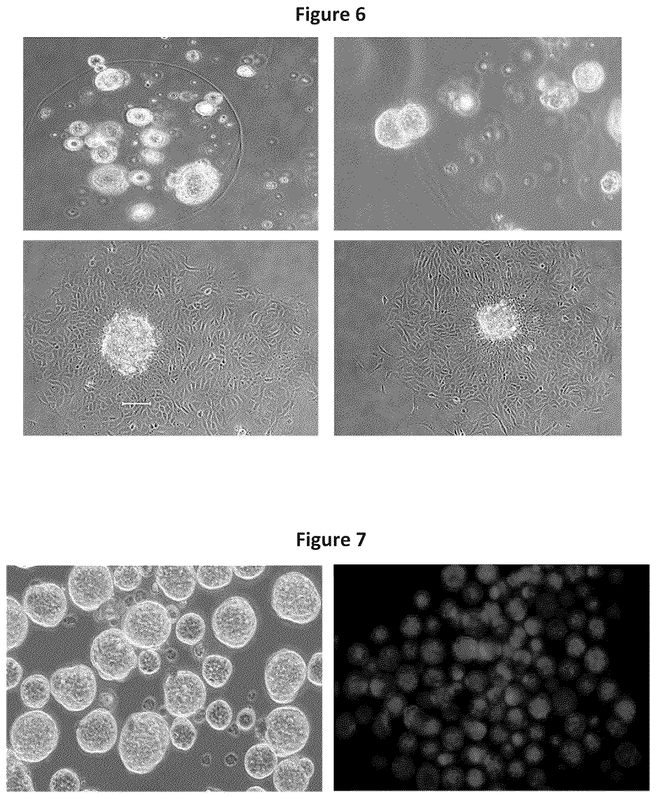

The STE-137 cells encapsulated in the alginate-gelatin microcarriers were able to proliferate and self-organize into spheroids with clear morphological characteristics. These spheroids exhibited a distinct spherical shape. Furthermore, the present inventors found that the microcarriers provided a suitable microenvironment for the growth and development of the spheroids, as they were able to support long-term culture and maintain the structural integrity of the spheroids over time. Results are shown in FIGS. 5A-B, 6 and 7.

### Example 6

### Encapsulation of Rainbow Trout (RT) Ovary Cells and GHB Cells

### Results

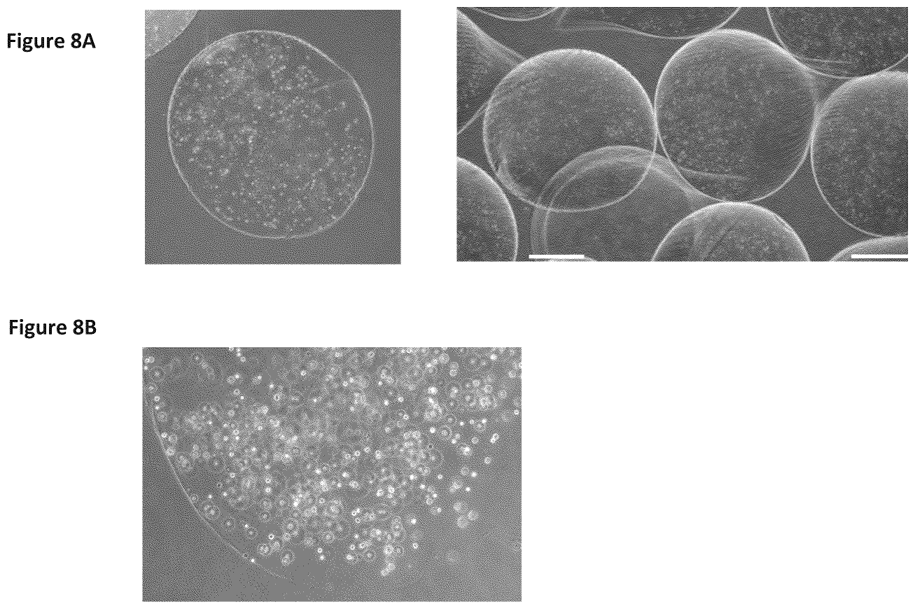

GHB or RT ovary cells were mixed with alginate-gelatin (1% and 2%, respectively) solution at a final concentration of 1×10⁶ cells per ml, and the mixture was formed into droplets that were cross-linked in a 1% calcium chloride solution to form hydrogel beads. These beads were then washed, transferred to a suitable culture medium, and maintained in an incubator at the appropriate temperature. FIG. 8A shows GHB cells on 1 day (left panel) and 5 days (right panel) post encapsulation within the beads, with a scale bar indicating 0.5 millimeters. FIG. 8B shows RT ovary cells 1 day post encapsulation within the beads, with a scale bar indicating 0.5 millimeters.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the Applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

1. A composition comprising edible hydrogel microcarriers-comprising cells, wherein said cells are distributed throughout said hydrogel microcarriers such that said hydrogel microcarriers are devoid of a cell-free core.

2. A method of producing edible hydrogel microcarriers-comprising cells, the method comprising:
(a) contacting a first solution comprising a cross-linker and a second solution comprising a polysaccharide, wherein at least one of said first solution and said second solution comprises cells so as to obtain a cross linked hydrogel comprising said cells;
(b) forming the hydrogel microcarriers-comprising cells from said cross linked hydrogel, such that the microcarriers are devoid of a cell-free core.

3. A composition comprising edible hydrogel microcarriers-comprising cells obtainable according to the method of claim 2.

4. (canceled)

5. A method of culturing cells, the method comprising incubating the composition of claim 1 in culture under conditions which allow proliferation and/or differentiation of said cells, thereby culturing the cells.

6. The composition of claim 1, wherein said hydrogel microcarriers are composed of a cross-linked polysaccharide.

7-8. (canceled)

9. The method of claim 2, wherein said polysaccharide is biodegradable or biocompatible.

10. (canceled)

11. The method of claim 2, wherein said polysaccharide is selected from the group of saccharides listed in Table 1.

12. The method of claim 2 further comprising a protein or peptide which serves as a structural protein composing said microcarrier.

13. The method of claim 2, being edible, optionally wherein said protein is gelatin or optionally wherein said protein or peptide is RGD-containing.

14-15. (canceled)

16. The method of claim 2, wherein said polysaccharide is alginate.

17. The method of claim 2, wherein said polysaccharide is alginate and said protein is gelatin.

18. The method of claim 2, further comprising removing said polysaccharide following said forming said edible hydrogel microcarriers-comprising cells.

19. The method of claim 18, wherein said removing is by using a chelator, e.g., EDTA or acid, e.g., citric acid.

20. The composition of claim 1, wherein said microcarriers are of an average size range of 500-1000 μm.

21. The composition of claim 1, wherein said cells comprise 1 or 2 cell types.

22. The composition of claim 1, wherein said cells are of an aquatic species.

23-25. (canceled)

26. The composition of claim 1, wherein said cells are stem cells or myogenic cells.

27-30. (canceled)

31. The method of claim 2, wherein said contacting is concomitantly or prior to with said forming.

32-42. (canceled)

43. A comestible comprising the composition of claim 1.

44-49. (canceled)
